(19) United States
(12) Patent Application Publication
(10) Pub. No.: US 2023/0374043 A1
(43) Pub. Date: Nov. 23, 2023

(54) Hexadeca Ammonium-Modified Phthalocyanine and Preparation Method and Use Thereof as Photodynamic Drug

(71) Applicant: Fuzhou University, Fuzhou (CN)

(72) Inventors: Jiandong Huang, Fuzhou (CN); Bingde Zheng, Fuzhou (CN); Meirong Ke, Fuzhou (CN); Biyuan Zheng, Fuzhou (CN)

(21) Appl. No.: 18/246,979

(22) PCT Filed: Feb. 3, 2021
(86) PCT No.: PCT/CN2021/074976

§ 371 Date: Mar. 28, 2023

(30) Foreign Application Priority Data
Oct. 16, 2020 (CN) 202011112741.0

(51) Int. Cl. C07F 3/06; A61K 41/00; A61P 31/04

(52) U.S. Cl. CPC C07F 3/06; A61K 41/0071; A61P 31/04

(57) ABSTRACT

The present disclosure provides a hexadeca amino- or hexadeca ammonium-modified zinc phthalocyanine and a preparation method and use thereof. In a zinc phthalocyanine structure, a 3-(dimethylamino)phenoxy substituent or a 3-(trimethylammonium) phenoxy substituent is located at peripheral positions α and β of a phthalocyanine ring. The zinc phthalocyanine complex has a high photosensitive activity and a high water solubility. The zinc phthalocyanine complex exists in the form of a monomer in water, which is conducive to exerting a photodynamic activity in water; meanwhile, the zinc phthalocyanine complex has an absorption spectrum red-shifted to 720 nm, which is located in a near-infrared region that is more conducive to penetrating human tissues. Therefore, the zinc phthalocyanine complex has higher tumor targeting ability and photodynamic tumor-suppression effect, and is scavenged quickly in vivo, which can be used for preparing a photosensitizer or a photodynamic drug or a photosensitive medicament.

### CROSS REFERENCE TO RELATED APPLICATION

The present application claims priority of Chinese Patent Application No. 202011112741.0 filed with the China National Intellectual Property Administration (CNIPA) on Oct. 16, 2020 and entitled “HEXADECA AMMONIUM-MODIFIED PHTHALOCYANINE AND PREPARATION METHOD AND USE THEREOF AS PHOTODYNAMIC DRUG”, which is incorporated herein by reference in its entirety.

### TECHNICAL FIELD

The present disclosure relates to the technical field of preparation of photodynamic drugs or photosensitizers, in particular to a hexadeca ammonium-modified phthalocyanine and a preparation method and use thereof as photodynamic drug.

### BACKGROUND

Photodynamic therapy (PDT) is a newly-developed efficient and palliative anticancer approach that has attracted enormous research interest in the past decade. Compared with traditional cancer treatments (such as surgery, chemotherapy, and radiotherapy), the PDT is non-invasive, has almost no side effects and drug resistance, and shows low systemic toxicity, high therapeutic efficiency, desirable tumor targeting, and broad-spectrum anti-cancer properties. At present, fungal infections, drug-resistant fungal diseases, and bacterial infections have emerged widely and become serious threats to public health, prompting the development of new antifungal/antibacterial drugs and therapeutic strategies. Photodynamic antibacterial method, as a new way of treating pathogenic fungi/bacteria, has a wide range of action, no drug resistance, and less damage to host tissues compared with traditional drug therapies.

Phthalocyanine is an aromatic heterocyclic ring with 18π electrons and composed of a four nitrogen atoms-bridged isoindole ring. The phthalocyanine mimics the biological value of a precursor molecule porphyrin, improves the spectrum and photochemical properties of porphyrin, and increases a specificity of the tumor targets. The phthalocyanine molecule has a cavity in the center that can chelate 63 different element ions, and has a strong absorption band in a near-infrared region to enhance the tissue penetration ability of light. In addition, there are many substitution sites on the phthalocyanine. Substitution can be conducted at non-peripheral (α) or peripheral (β) positions of the phthalocyanine macromolecules on the one hand, and axial substitution can also be conducted through a central metal on the other hand, contributing to applicability of these molecules to a variety of different scientific fields.

A key to both the PDT and the photodynamic antibacterial method lies in the photosensitizers. As a second-generation photosensitizer, phthalocyanine has many advantages in the field of PDT. For example: (1) Compared with the maximum absorption wavelength of porphyrin (400 nm to 600 nm), the phthalocyanine has a maximum absorption wavelength of greater than 670 nm and a high extinction coefficient (greater than 10⁵ M⁻¹ cm⁻¹), indicating strong photosensitization ability. (2) Phthalocyanine has a structure that is easy to modify, with a desirable stability. (3) Phthalocyanine has low dark toxicity, can make radiation penetrate tissues to a greater extent, and can avoid a visible light region of 400 nm to 600 nm as much as possible. Accordingly, the phthalocyanine can significantly reduce the phototoxicity of sunlight on the skin. (4) Phthalocyanine does not have drug resistance.

As a result, based on these excellent properties, the phthalocyanine has been widely used in the field of PDT. However, the currently reported biologically-active phthalocyanine complexes still have deficiencies, such as easy aggregation in water, or complex synthetic routes, or poor targeting ability, or therapeutic window being not in the near-infrared region, or slow metabolism, or more liver retention, which all need further improvement. In addition, due to the potential huge economic and social value, a huge range of applications, and the refinement of treatment lesions in photosensitizers and PDT, it is also necessary to prepare more advantageous phthalocyanine complexes as drug candidates.

### SUMMARY

An objective of the present disclosure is to provide a hexadeca amino- or hexadeca ammonium-modified zinc phthalocyanine and a preparation method and use thereof as a photodynamic drug. In the present disclosure, the zinc phthalocyanine complex has a high photodynamic activity, easily-available raw materials, a easy preparation method, is not easy to aggregate in physiological systems, and has a high stability, a spectrum significantly red-shifted to a near-infrared region, a desirable targeting ability, and fast metabolism in vivo. Therefore, the phthalocyanine complex can be applied to PDT or a photodynamic antibacterial method.

To achieve the above objective, the present disclosure provides the following technical solutions:

The present disclosure provides a hexadeca ammonium-modified zinc phthalocyanine, as a zinc phthalocyanine complex tetrasubstituted at peripheral positions α and β, and including a 3-(dimethylamino)phenoxy substituent, where the substituent is separately located at the peripheral positions α and β of a phthalocyanine ring, namely positions 1, 2, 3, 4, 8, 9, 10, 11, 15, 16, 17, 18, 22, 23, 24, and 25; the zinc phthalocyanine complex is named as a 1,2,3,4,8,9,10,11,15,16,17,18,22,23,24,25-hexadeca[3-(dimethylamino)phenoxy]zinc phthalocyanine complex, or a hexadeca-[3-(dimethylamino)phenoxy]zinc phthalocyanine complex; and the hexadeca amino-modified zinc phthalocyanine has a structural formula as follows:

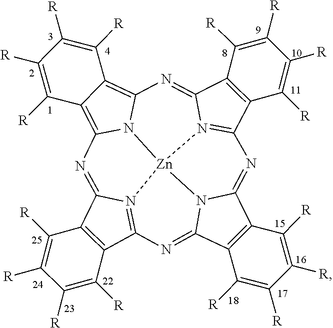

in the formula,

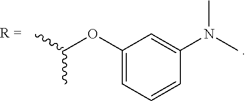 [SMILES: *Oc1cccc(N(C)C)c1]

The present disclosure further provides a preparation method of the hexadeca amino-modified zinc phthalocyanine, including the following steps:
1) preparation of 3,4,5,6-tetrakis[3-(dimethylamino)phenoxy]phthalonitrile: conducting a reaction by stirring at 110° C. for 18 h to 20 h with 3,4,5,6-tetrachlorophthalonitrile and N,N-dimethyl-3-aminophenol as a reactant using N,N-dimethylformamide as a solvent in the presence of potassium carbonate and nitrogen protection; monitoring the reaction by thin-layer chromatography, and terminating the reaction when the 3,4,5,6-tetrachlorophthalonitrile is consumed; and conducting purification by extraction, chromatography, or recrystallization to obtain the 3,4,5,6-tetrakis[3-(dimethylamino)phenoxy]phthalonitrile; and
2) preparation of the hexadeca amino-modified zinc phthalocyanine: using the 3,4,5,6-tetrakis[3-(dimethylamino)phenoxy]phthalonitrile as a raw material and methanol as a solvent, and adding zinc acetate to obtain a mixture; conducting a reaction by stirring on the mixture at 65° C. to 85° C. for 5 h to 6 h with 1,8-diazabicyclo[5.4.0]undec-7-ene as a catalyst; monitoring a reaction endpoint by the thin-layer chromatography to generate the hexadeca amino-modified zinc phthalocyanine; and conducting purification by a solvent method or the chromatography to obtain a target product.

Further, in step 1), the 3,4,5,6-tetrachlorophthalonitrile and the N,N-dimethyl-3-aminophenol are added at a molar ratio of 1:(5.5-6.0); 5 mL to 6 mL of the N,N-dimethylformamide is used for per mmol of the 3,4,5,6-tetrachlorophthalonitrile, and 7.5 mmol to 8 mmol of the potassium carbonate is used for per mmol of the 3,4,5,6-tetrachlorophthalonitrile;

in step 2), the 3,4,5,6-tetrakis[3-(dimethylamino)phenoxy]phthalonitrile and the zinc acetate are added at a molar ratio of (2-4):1; 10 mL to 15 mL of the methanol is used for per mmol of the 3,4,5,6-tetrakis[3-(dimethylamino)phenoxy]phthalonitrile, and 3 mL to 4 mL of the 1,8-diazabicyclo[5.4.0]undec-7-ene is used for per mmol of the 3,4,5,6-tetrakis[3-(dimethylamino)phenoxy]phthalonitrile.

The present disclosure further provides a hexadeca ammonium-modified zinc phthalocyanine, as a zinc phthalocyanine complex tetrasubstituted at peripheral positions α and β, and including a 3-(trimethylammonium) phenoxy substituent, where the substituent is separately located at the peripheral positions α and β of a phthalocyanine ring, namely positions 1, 2, 3, 4, 8, 9, 10, 11, 15, 16, 17, 18, 22, 23, 24, and 25; the zinc phthalocyanine complex is named as a 1,2,3,4,8,9,10,11,15,16,17,18,22,23,24,25-hexadeca[3-(trimethylammonium) phenoxy]zinc phthalocyanine complex, or a hexadeca-[3-(trimethylammonium) phenoxy]zinc phthalocyanine complex; and the hexadeca ammonium-modified zinc phthalocyanine has a structural formula as follows:

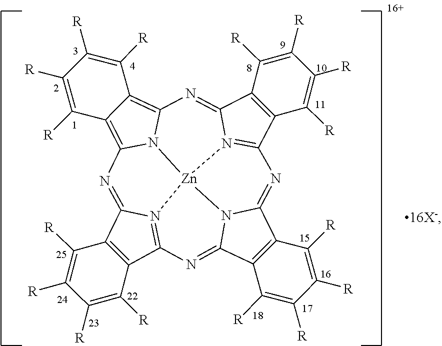

in the formula,

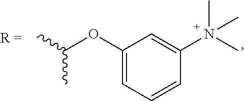

and X is selected from the group consisting of I and Br.

The present disclosure further provides a preparation method of the hexadeca ammonium-modified zinc phthalocyanine, including the following steps:
1) preparation of 3,4,5,6-tetrakis[3-(dimethylamino)phenoxy]phthalonitrile: conducting a reaction by stirring at 110° C. for 18 h to 20 h with 3,4,5,6-tetrachlorophthalonitrile and N,N-dimethyl-3-aminophenol as a reactant using N,N-dimethylformamide as a solvent in the presence of potassium carbonate and nitrogen protection; monitoring the reaction by thin-layer chromatography, and terminating the reaction when the 3,4,5,6-tetrachlorophthalonitrile is consumed; and conducting purification by extraction, chromatography, or recrystallization to obtain the 3,4,5,6-tetrakis[3-(dimethylamino)phenoxy]phthalonitrile; and
2) preparation of the hexadeca amino-modified zinc phthalocyanine: using the 3,4,5,6-tetrakis[3-(dimethylamino)phenoxy]phthalonitrile as a raw material and methanol as a solvent, and adding zinc acetate to obtain a mixture; conducting a reaction by stirring on the mixture at 65° C. to 85° C. for 5 h to 6 h with 1,8-diazabicyclo[5.4.0]undec-7-ene as a catalyst; monitoring a reaction endpoint by the thin-layer chromatography, and conducting purification by a solvent method or the chromatography to obtain the hexadeca amino-modified zinc phthalocyanine; and
3) preparation of hexadeca ammonium-modified zinc phthalocyanine: using the hexadeca amino-modified zinc phthalocyanine prepared in step 2) as a raw material and chloroform or N,N-dimethylformamide as a solvent, and adding methyl iodide or methyl bromide to obtain a mixture; conducting a reaction by stirring on the mixture at 15° C. to 35° C. for 6 h to 12 h to generate the hexadeca ammonium-modified zinc phthalocyanine, and conducting purification by the solvent method or the chromatography to obtain a target product.

Further, in step 1), the 3,4,5,6-tetrachlorophthalonitrile and the N,N-dimethyl-3-aminophenol are added at a molar ratio of 1:(5.5-6.0); 5 mL to 6 mL of the N,N-dimethylformamide is used for per mmol of the 3,4,5,6-tetrachlorophthalonitrile, and 7.5 mmol to 8 mmol of the potassium carbonate is used for per mmol of the 3,4,5,6-tetrachlorophthalonitrile;

in step 2), the 3,4,5,6-tetrakis[3-(dimethylamino)phenoxy]phthalonitrile and the zinc acetate are added at a molar ratio of (2-4):1; 10 mL to 15 mL of the methanol is used for per mmol of the 3,4,5,6-tetrakis[3-(dimethylamino)phenoxy]phthalonitrile, and 3 mL to 4 mL of the 1,8-diazabicyclo[5.4.0]undec-7-ene is used for per mmol of the 3,4,5,6-tetrakis[3-(dimethylamino)phenoxy]phthalonitrile; and

in step 3), 0.8 mL to 2 mL of the methyl iodide or the methyl bromide is used for per 0.1 mmol of the hexadeca amino-modified zinc phthalocyanine, and 1.0 mL to 2.0 mL of the N,N-dimethylformamide or 1.0 mL to 2.0 mL of the chloroform is used for per 0.1 mmol of the hexadeca amino-modified zinc phthalocyanine.

In the present disclosure, the hexadeca amino-modified zinc phthalocyanine and the hexadeca ammonium-modified zinc phthalocyanine can be used to prepare a photosensitizer or a photodynamic drug or a photosensitive medicament. The photosensitive medicament, or the photosensitizer for short, or called a photosensitive drug preparation, is also called the photodynamic drug and can be used for PDT, photodynamic diagnosis, or photodynamic disinfection. The PDT can be the PDT of malignant tumors, or the PDT of leukemia based on bone marrow purification in vitro, or the PDT of non-cancer diseases, such as fungal infections, bacterial infections, oral diseases, macular degeneration-based eye diseases, arteriosclerosis, trauma infections, skin diseases, and virus infections. The photodynamic disinfection may include photodynamic sterilization and purification of blood or a blood derivative, photodynamic sterilization and disinfection of water, and photodynamic disinfection of a medical or household appliance.

The present disclosure further provides use of the hexadeca amino-modified zinc phthalocyanine or the hexadeca ammonium-modified zinc phthalocyanine in PDT, photodynamic diagnosis, and photodynamic disinfection, where a suitable light source is required, and the suitable light source can be provided by an ordinary light source connected with a suitable filter, or by a laser with a specific wavelength; the suitable light source has a wavelength of 680 nm to 730 nm.

A method for preparing a photosensitive medicament with the zinc phthalocyanine includes: dissolving the hexadeca amino-modified zinc phthalocyanine or the hexadeca ammonium-modified zinc phthalocyanine in water or a mixed solution of water and other substances, to obtain the photosensitive medicament with a certain concentration (where a concentration of the zinc phthalocyanine complex is not higher than that in a saturated solution of the zinc phthalocyanine complex); and adding an additive including an antioxidant, a buffer, and an isotonic agent to a prepared medicament solution to maintain a chemical stability and a biocompatibility of the photosensitive medicament.

Further, the mixed solution has not greater than 10% of the other substances by mass fraction; and the other substances are one or a mixture of two or more selected from the group consisting of a castor oil derivative, dimethyl sulfoxide (DMSO), ethanol, glycerol, N,N-dimethylformamide, polyethylene glycol 300 to polyethylene glycol 3000, cyclodextrin, glucose, Tween, and polyethylene glycol monostearate.

Further, for a pharmaceutical preparation for topical administration, a preparation method of the photosensitive medicament includes: dissolving the hexadeca amino-modified zinc phthalocyanine or the hexadeca ammonium-modified zinc phthalocyanine in a permeable solvent, or injecting the hexadeca amino-modified zinc phthalocyanine or the hexadeca ammonium-modified zinc phthalocyanine into an ointment, a lotion, or a gel and stirring uniformly; the permeable solvent is a DMSO aqueous solution with a mass fraction of 5 wt % to 35 wt %.

Beneficial effects and outstanding advantages of the present disclosure are as follows:
(1) In the hexadeca amino-modified zinc phthalocyanine or the hexadeca ammonium-modified zinc phthalocyanine provided by the present disclosure, the peripheral positions α and β of the phthalocyanine ring are simultaneously replaced by amino groups or ammonium groups, resulting in a clear structure, no positional isomers, and easy preparation processes.
(2) In the hexadeca amino-modified zinc phthalocyanine or the hexadeca ammonium-modified zinc phthalocyanine provided by the present disclosure, the preparation method has a reasonable and feasible route, easy access to synthetic raw materials, and easy industrialization.
(3) In the hexadeca amino-modified zinc phthalocyanine or the hexadeca ammonium-modified zinc phthalocyanine provided by the present disclosure, the product has a red-shifted maximum absorption wavelength to 720 nm, and a high molar absorption coefficient (up to 10⁵ orders of magnitude), such that a tissue penetration ability of the action spectrum is further improved, which is extremely beneficial for PDT and photodynamic diagnosis.
(4) In the hexadeca amino-modified zinc phthalocyanine or the hexadeca ammonium-modified zinc phthalocyanine provided by the present disclosure, the product can exist in the form of a monomer in an aqueous system without any surfactant or solubilization by organic solvent, such that the photodynamic activity can be greatly improved, showing a potential advantage as a photodynamic drug. The hexadeca amino-modified zinc phthalocyanine or the hexadeca ammonium-modified zinc phthalocyanine has a photodynamic activity for human liver cancer cells HepG2 that is significantly higher than that of other similar compounds, such as tetrakis-α-(2,4,6-tris(N,N,N-trimethylammonium methyl)-phenoxy)zinc phthalocyanine dodecaiodate.
(5) In the hexadeca amino-modified zinc phthalocyanine or the hexadeca ammonium-modified zinc phthalocyanine provided by the present disclosure, the product has a desirable tumor targeting ability. Meanwhile, the product has a high scavenge rate in vivo (which is faster than other phthalocyanine photosensitizers), showing a high biological safety.
(6) In the hexadeca amino-modified zinc phthalocyanine or the hexadeca ammonium-modified zinc phthalocyanine provided by the present disclosure, the product has a high photodynamic tumor-suppression effect, with a tumor inhibition rate of not less than 90%.
(7) In the hexadeca amino-modified zinc phthalocyanine or the hexadeca ammonium-modified zinc phthalocyanine provided by the present disclosure, the product has an excellent photodynamic antibacterial activity, with an IC₉₀ value reaching an nM level, which is 86 times higher than the IC₉₀ value (5.9 μM) of a commonly-used photosensitizer MB.

### BRIEF DESCRIPTION OF THE DRAWINGS

FIG. 1 shows an ultraviolet-visible absorption spectrum (at 4 μM) of zinc phthalocyanine complexes obtained in Examples 1 to 8 in water.

### DETAILED DESCRIPTION OF THE EMBODIMENTS

The present disclosure is further described below with reference to the accompanying drawings and examples.

### EXAMPLE 1

A synthesis method of a 2,3-bis[3-(dimethylamino)phenoxy]zinc phthalocyanine complex included the following steps:
1) 5.08 mmol of 4,5-dichlorophthalonitrile, 15.23 mmol of N,N-dimethyl-3-aminophenol, and 20.3 mmol of potassium carbonate were placed in a 100 mL double-necked bottle, fully dissolved with 20 mL of anhydrous N,N-dimethylformamide, and subjected to a reaction by stirring at 90° C. for about 18 h under the protection of nitrogen. After the reaction was completed, the potassium carbonate was removed by suction filtration, a reaction solution was concentrated slightly, poured into 300 mL of ice water, and then extracted three times with ethyl acetate, and an organic layer was collected. The organic layer was washed three times with a 1 M sodium hydroxide solution and a saturated sodium chloride separately, and a new organic layer was collected. The new organic layer was dried with anhydrous sodium sulfate, subjected to suction filtration, and a collected filtrate was subjected to rotary evaporation to dryness. An obtained product was dissolved with the ethyl acetate, passed through a silica gel column with a mixture of dichloromethane: ethyl acetate=100:1 as an eluent, and a collected first bright yellow band was subjected to rotary evaporation to dryness. A resulting product was recrystallized with methanol, subjected to suction filtration, and dried under vacuum to obtain 4,5-bis[3-(dimethylamino)-phenoxy]phthalonitrile as an orange-red product with a yield of 93%.

The characterization data of the product were as follows: ¹H NMR (400 MHz, DMSO-d6, ppm): δ7.61(s,2H, Ar—H), 7.23 (t, J=8.0 Hz, 2H, Ar—H), 6.59 (dd, J=8.4, 2.0 Hz, 2H, Ar—H), 6.41 (s, 2H, Ar—H), 6.35 (d, J=8.0 Hz, 2H, Ar—H), 2.90 (s, 12H, CH₃).

HRMS (ESI): m/z calcd for C₂₄H₂₃N₄O₂ [M+H]+, 399.1816; found 399.1814. calcd for C₂₄H₂₂N₄O₂Na[M+Na]³⁰ , 421.1635; found 421.1633.
2) 1 mmol of the 4,5-bis[3-(dimethylamino)-phenoxy]phthalonitrile, 6 mmol of phthalonitrile, and 0.5 mmol of anhydrous zinc acetate were placed in a 100 mL double-necked bottle, added with 20 mL of n-pentanol, and gradually heated to 90° C. under nitrogen protection to dissolve fully to obtain a mixture; 0.5 mL of 1,8-diazabicyclo[5.4.0]undec-7-ene was added to the mixture, and a reaction was conducted at 135° C. for about 12 h. After the reaction, a product was subjected to rotary evaporation to dryness. A product was dissolved in a small amount of N,N-dimethylformamide, passed through a silica gel column, and a green phthalocyanine band was collected with dichloromethane as an eluent, and then subjected to rotary evaporation to dryness. A resulting product was dissolved in a small amount of tetrahydrofuran, passed through an X3-type gel, and a first green phthalocyanine band collected was subjected to rotary evaporation to dryness. An obtained product was dissolved in a small amount of dichloromethane, passed through a silica gel column, and a green phthalocyanine band was collected with a mixture of dichloromethane: methanol=20:1, and then subjected to rotary evaporation to dryness. A resulting product was repeatedly washed with n-hexane to remove yellow impurities, and dried under vacuum to obtain 220 mg of a target zinc phthalocyanine complex, with a yield of 26%; a structural formula was as follows:

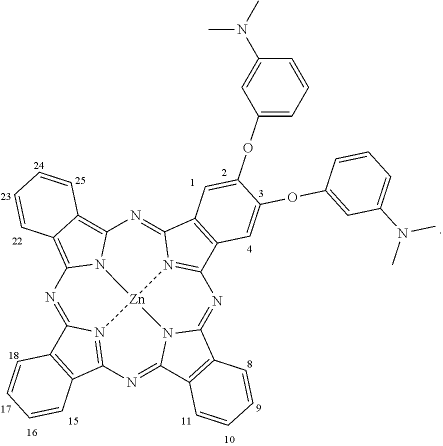

The characterization data of the product were as follows: ¹HNMR (400 MHz,DMSO-d6, ppm): δ9.38-9.00 (m, 6H, Pc—H_{α}), 8.79-8.60 (m, 3H, Pc—H_{α}, Pc—H_{β}), 8.30-8.06 (m, 5H,Pc—H_{β}), 7.45-7.29 (m, 3H, Ar—H), 6.72 (d, J=16 Hz,5H, Ar—H), 3.01 (s, 12H,CH₃).

HRMS (ESI): m/z calcd for C₄₈H₃₅N₁₀O₂Zn [M+H]⁺, 847.2230; found 847.2237.

IR (KBr, cm−1): 3432.25 (Ar—H); 2925.07 (—CH₃); 1653.88, 1524.30, 1498.40, 1430.17 (C═C, C═N—); 1242.47 (Ar—O—Ar); 1080.81, 1000.97, 911.99, 760.79, 732.88 (Ar—H).

### EXAMPLE 2

A synthesis method of a 2,3-bis[3-(trimethylammonium)phenoxy]zinc phthalocyanine diiodate included the following steps:
1) according to the method in Example 1, the 2,3-bis[3-(dimethylamino)phenoxy]zinc phthalocyanine complex was prepared; and
2) 0.3 mmol of the 2,3-bis[3-(dimethylamino)phenoxy]zinc phthalocyanine complex and 2 mL of methyl iodide were added into 5 mL of chloroform, and subjected to a reaction by stirring at 25° C. for 6 h; after the reaction, a reaction solution was subjected to suction filtration, washed with chloroform until a filtrate was colorless, and a filter cake was collected and dried under vacuum to obtain 25.6 mg of a target product, with a yield of 77%; a structural formula was as follows:

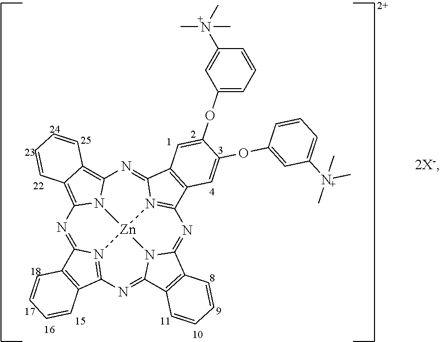

in the formula, X is I.

The characterization data of the product were as follows: ¹H NMR (400 MHz, DMSO-d6, ppm): δ9.57-8.81 (m, 8H, Pc—H_{α}), 8.40-8.00 (m, 7H, Pc—H_{β}, Ar—H), 7.97-7.55 (m, 7H,Ar—-H), 3.74 (s, 18H, CH₃).

HRMS (ESI): m/z calcd for C₅₀H₄₀N₁₀O₂Zn [M-2I]²⁺, 438.1308; found 438.1314.

IR (KBr, cm⁻¹): 3423.70 (Ar—H); 2923.91, 2853.19 (—CH₃); 1605.35, 1560.45, 1459.15, 1405.90 (C═C, C═N—); 1223.16 (Ar—O—Ar); 1088.14, 931.46, 735.73 (Ar—H).

### EXAMPLE 3

A synthesis method of a 1,2,3,4-tetrakis[3-(dimethylamino)phenoxy]zinc phthalocyanine complex included the following steps:
1) 3.76 mmol of 3,4,5,6-tetrachlorophthalonitrile, 22.56 mmol of N,N-dimethyl-3-aminophenol, and 30.09 mmol of potassium carbonate were placed in a 100 mL double-necked bottle, fully dissolved with 20 mL of anhydrous N,N-dimethylformamide, and subjected to a reaction by stirring at 110° C. for about 18 h under the protection of nitrogen. After the reaction was completed, the potassium carbonate was removed by suction filtration, a reaction solution was concentrated slightly, poured into 300 mL of ice water, and then extracted three times with ethyl acetate, and an organic layer was collected. The organic layer was washed three times with a 1 M sodium hydroxide solution and a saturated sodium chloride separately, and a new organic layer was collected. The new organic layer was dried with anhydrous sodium sulfate, subjected to suction filtration, and a filtrate was subjected to rotary evaporation to dryness. An obtained product was dissolved with the ethyl acetate, passed through a silica gel column with a mixture of dichloromethane: ethyl acetate=10:1 as an eluent, and a collected first bright yellow band was subjected to rotary evaporation to dryness. A resulting product was recrystallized with methanol, subjected to suction filtration, and a filter cake was collected and dried under vacuum to obtain 0.83 g of 3,4,5,6-tetrakis[3-(dimethylamino)phenoxy]phthalonitrile as a bright yellow product with a yield of 66%.

The characterization data of the product were as follows: ¹HNMR (400 MHz,DMSO-d6, ppm): δ7.08 (t,J=8.2 Hz,2H,Ar—H),6.97(t,J=8.0 Hz,2H,Ar—H),6.45(d,J=7.6Hz,2H,Ar—H), 6.37(d,J=8.0 Hz,2H,Ar—H),6.29(d,J=8.0 Hz,2H,Ar—H),6.14(s,2H,Ar—H),6.02(d,J=7.2Hz,2H, Ar—H),5.75(s,2H, Ar—H),2.84(s,12H),2.75 (s, 12H).

HRMS (ESI): m/z calcd for C₄₀H₄₁N₆O₄[M+H]+, 669.3184; found 669.3173.
2) 0.3 mmol of the 3,4,5,6-tetrakis[3-(dimethylamino)phenoxy]phthalonitrile, 6 mmol of phthalonitrile, and 0.5 mmol of anhydrous zinc acetate were dissolved with 20 mL of n-pentanol, and gradually heated to 95° C. under nitrogen protection to dissolve fully to obtain a mixture; 1 mL of 1,8-diazabicyclo[5.4.0]undec-7-ene was added to the mixture, and a reaction was conducted at 135° C. for about 12 h. After the reaction, a product was subjected to rotary evaporation to remove the organic solvent. A product was dissolved in a small amount of N,N-dimethylformamide, passed through a silica gel column, and a yellow-green phthalocyanine band was collected with dichloromethane as an eluent, and then subjected to rotary evaporation to dryness. A resulting product was dissolved in a small amount of tetrahydrofuran, passed through an X3-type gel, and a green phthalocyanine band collected was subjected to rotary evaporation to dryness. An obtained product was dissolved in a small amount of dichloromethane, passed through a silica gel column, and a green phthalocyanine band was collected with a mixture of dichloromethane: methanol=40:1, and then subjected to rotary evaporation to dryness. A resulting product was repeatedly washed with n-hexane to remove yellow impurities, and dried to obtain 80 mg of a target zinc phthalocyanine complex, with a yield of 12%; a structural formula was as follows:

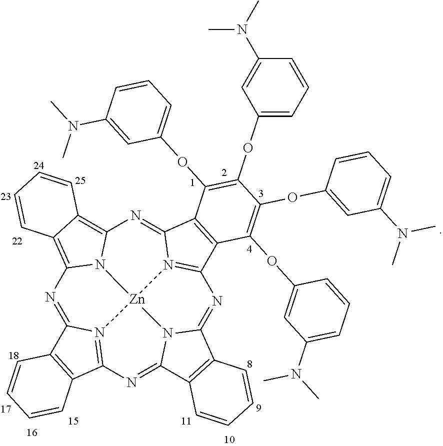

The characterization data of the product were as follows: ¹ NMR (400 MHz, DMSO-d6, ppm): δ9.26 (s,3H, Pc—H_{α}), 8.95 (s, 1H, Pc—H_{α}), 8.19-7.94 (m, 6H, Pc—H_{α}, Pc—H_{β}), 7.23-6.90(m,8H,Pc—H_{β},Ar—H), 6.62-6.34(m,8H,Ar—H),6.27(s,2H,Ar—H),3.13-2.61(m, 24H, CH₃).

HRMS (ESI): m/z calcd for C₆₄H₅₃N₁₂O₄Zn [M+H]+, 1117.3599; found 1117.3639.

IR (KBr, cm−1): 3431.04 (Ar—H); 2925.67 (—CH₃); 1734.90, 1617.30, 1497.87, 1400.50 (C═C, C═N—); 1273.26 (Ar—O—Ar); 1088.97, 1031.20, 1000.05, 640.18, 611.00, 571.50, 535.03 (Ar—H).

### EXAMPLE 4

A synthesis method of a 1,2,3,4-tetrakis[3-(dimethylammonium)phenoxy]zinc phthalocyanine tetraiodate included the following steps:
1) according to the method in Example 3, the 1,2,3,4-tetrakis[3-(dimethylamino)phenoxy]zinc phthalocyanine complex was prepared; and
2) 0.36 mmol of the 1,2,3,4-tetrakis[3-(dimethylamino)phenoxy]zinc phthalocyanine complex and 3 mL of methyl iodide were added into 5 mL of chloroform, and subjected to a reaction by stirring at 25° C. for 12 h; after the reaction, a reaction solution was subjected to suction filtration, washed with dichloromethane, ethyl acetate, and acetone separately until a filtrate was colorless, and a filter cake was collected and dried under vacuum to obtain 40 mg of a target product, with a yield of 66%; a structural formula was as follows:

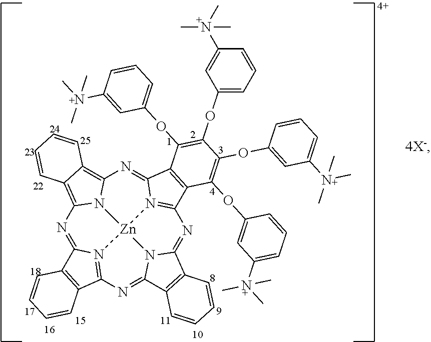

in the formula, X is I.

The characterization data of the product were as follows: ¹HNMR (400 MHz,DMSO-d6, ppm): δ9.52-9.35 (m, 2H, Pc—H_{α}), 8.69 (d, J=7.6 Hz,1H, Pc—H_{α}), 8.48-8.07 (m, 5H,Pc—H_{α},Pc—H_{β}), 7.77-7.72(m,4H,Pc—H_{β}),7.72-7.24(m,14H,Ar—H),6.96(s,2H, Ar—H), 3.74-3.46 (m, 36H, CH₃).

HRMS (ESI): m/z calcd for C₆₈H₆₄N₁₂O₄Zn [M-4I]⁴⁺, 294.1111; found 294.1100. calcd for C₆₈H₆₄N₁₂O₄ZnI [M-3I]³⁺, 434.4498; found 434.4483.

IR (KBr, cm−1): 3435.08 (Ar—H); 3022.26 (—CH₃); 1607.48, 1524.27, 1490.01, 1435.16, 1404.66, 1334.14 (C═C, C═N—); 1220.27 (Ar—O—Ar);1117.38, 1089.49, 1058.91, 1005.11, 967.42, 931.49, 751.44, 685.12, 609.42, 577.61, 496.06 (Ar—H).

### EXAMPLE 5

A synthesis method of a 2,3,9,10,16,17,23,24-octa[3-(dimethylamino)phenoxy]zinc phthalocyanine complex, or called an octa-β-[3-(dimethylamino)phenoxy]zinc phthalocyanine complex included the following steps:
1) according to step 1) of the method in Example 1, the 4,5-bis[3-(dimethylamino)-phenoxy]phthalonitrile was prepared; and
2) 1 mmol of the 4,5-bis[3-(dimethylamino)-phenoxy]phthalonitrile and 0.5 mmol of anhydrous zinc acetate were placed in a 100 mL double-necked bottle, added with 3 mL of methanol, and gradually heated to 85° C. under nitrogen protection to dissolve fully to obtain a mixture; 0.5 mL of 1,8-diazabicyclo[5.4.0]undec-7-ene was added to the mixture, and a reaction was conducted for about 5 h. After the reaction, a resulting reaction solution was poured into a large amount of methanol to precipitate a precipitate, the precipitate was subjected to suction filtration, and then washed with methanol, distilled water, and saturated brine separately, and a filter cake was collected and dried under vacuum. An obtained product was dissolved in a small amount of dichloromethane, passed through a silica gel column, and a green phthalocyanine band was collected with a mixture of dichloromethane: methanol=10:1, and then subjected to rotary evaporation to dryness. A resulting product was dissolved with a small amount of dichloromethane and precipitated with a large amount of methanol to obtain a precipitate, the precipitate was subjected to suction filtration, and then washed with methanol, water, and saturated brine separately, and then dried under vacuum. An obtained product was dissolved with a small amount of N,N-dimethylformamide, washed with a large amount of water to obtain a precipitate, the precipitate was subjected to suction filtration, washed with water, and then dried under vacuum. A resulting product was dissolved in a small amount of dichloromethane, passed through a silica gel column, and a green phthalocyanine band was collected with a mixture of dichloromethane: methanol=10:1, and then subjected to rotary evaporation to dryness. An obtained product was repeatedly washed with n-hexane and a hot methanol solution separately to remove yellow impurities, and dried under vacuum to obtain 180 mg of a target zinc phthalocyanine complex, with a yield of 10%; a structural formula was as follows:

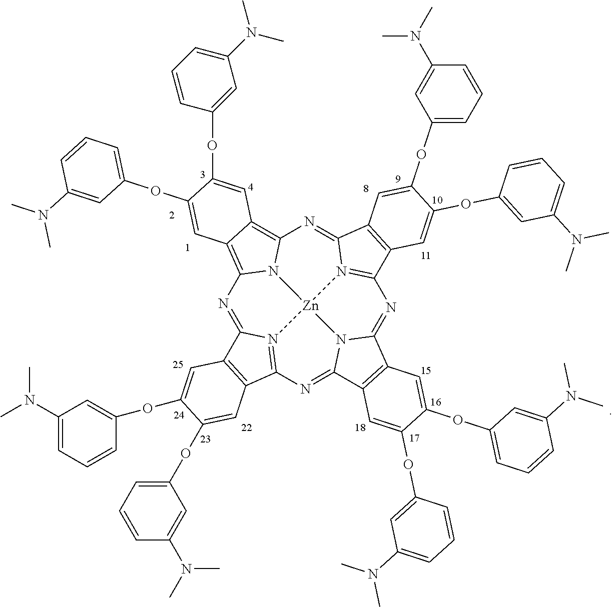

The characterization data of the product were as follows: ¹HNMR (400 MHz,CDCl₃-d+adroppyridine-d5, ppm): δ9.00 (s, 8H, Pc—H_{α}), 8.02 (s, 2H, Ar—H), 7.22 (s, 6H,Ar—H), 6.81-6.28 (m, 24H, Ar—H), 3.10-2.76 (m, 48H, CH₃).

HRMS (ESI): m/z calcd for C₉₆H₈₉N₁₆O₈Zn [M+H]⁺, 1659.6352; found1659.6397.

IR (KBr, cm−1): 3430.57 (Ar—H); 2916.99 (—CH₃); 1617.26, 1503.49, 1400.50 (C═C, C═N—); 1276.77 (Ar—O—Ar); 1059.10, 1029.97, 1000.21, 969.79, 669.15, 612.78, 576.84, 526.18, 487.97 (Ar—H).

### EXAMPLE 6

A synthesis method of a 2,3,9,10,16,17,23,24-octa[3-(trimethylammonium)phenoxy]zinc phthalocyanine octaiodate, or called an octa-β-[3-(trimethylammonium)phenoxy]zinc phthalocyanine octaiodate included the following steps:
1) according to the method in Example 5, the octa-β-[3-(dimethylamino)phenoxy]zinc phthalocyanine complex was prepared; and
2) 0.12 mmol of the octa-β-[3-(dimethylamino)phenoxy]zinc phthalocyanine complex and 3 mL of methyl iodide were added into 5 mL of chloroform, and subjected to a reaction by stirring at 25° C. for 6 h; after the reaction, a reaction solution was subjected to suction filtration, washed with dichloromethane, ethyl acetate, and acetone separately until a filtrate was colorless, and a filter cake was collected and dried under vacuum to obtain 19 mg of a target product, with a yield of 56%; a structural formula was as follows:

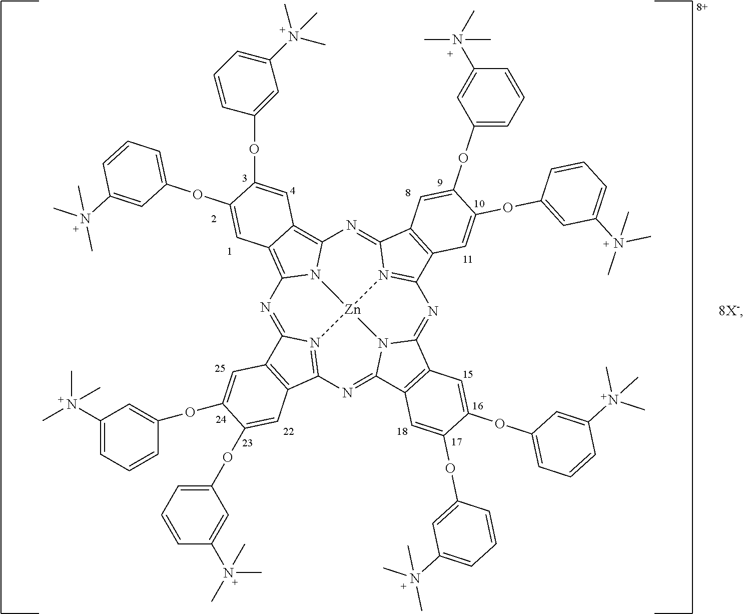

in the formula, X is I.

The characterization data of the product were as follows: 1H NMR (400 MHz, DMSO-d6, ppm): δ9.23 (s,6H, Pc—H_{α}), 9.10 (s, 1H, Pc—H_{α}), 8.93 (s, 1H, Pc—H_{α}), 7.98 (s, 7H, Ar—H),7.86-7.62(m,15H,Ar—H),7.45(d,J=8.4 Hz,6H,Ar—H),6.53(s,4H,Ar—H),3.34 (s, 72H, CH₃).

HRMS (ESI): m/z calcd for C₁₀₄H₁₁₂N₁₆O₈Zn [M-8I]⁸⁺, 222.3515; found 222.3509.

IR (KBr, cm−1): 3445.41 (Ar—H); 1635.04, 1516.00, 1397.854 (C═C, C═N—).

### EXAMPLE 7

A synthesis method of a 1,2,3,4,8,9,10,11,15,16,17,18,22,23,24,25-hexadeca[3-(dimethylamino)phenoxy]zinc phthalocyanine complex, or called a hexadeca-[3-(dimethylamino)phenoxy]zinc phthalocyanine complex included the following steps:
1) according to step 1) of the method in Example 3, the 3,4,5,6-tetrakis[3-(dimethylamino)phenoxy]phthalonitrile was prepared; and
2) 0.3 mmol of the 3,4,5,6-tetrakis[3-(dimethylamino)phenoxy]phthalonitrile and 0.15 mmol of anhydrous zinc acetate were dissolved with 3 mL of methanol, and gradually heated to 85° C. under nitrogen protection to dissolve fully to obtain a mixture; 1 mL of 1,8-diazabicyclo[5.4.0]undec-7-ene was added to the mixture, and a reaction was conducted for about 5 h. After the reaction, a product was subjected to rotary evaporation to remove the organic solvent. A resulting product was dissolved with a small amount of dichloromethane and precipitated with a large amount of methanol to obtain a precipitate, the precipitate was subjected to suction filtration, and then washed with methanol, distilled water, and saturated brine separately, and then dried under vacuum. An obtained product was dissolved in a small amount of dichloromethane, passed through a silica gel column, and a yellow-green phthalocyanine band was collected with a mixture of dichloromethane: methanol=10:1, and then subjected to rotary evaporation to dryness. A resulting product was dissolved in a small amount of tetrahydrofuran, passed through an X3-type gel, and a yellow-green phthalocyanine band collected was subjected to rotary evaporation to dryness. A resulting product was dissolved with a small amount of dichloromethane and precipitated with a large amount of methanol to obtain a precipitate, the precipitate was washed with methanol, water, and saturated brine separately, where the dissolution and precipitation were repeated 3 times. An obtained product was dissolved with a small amount of N,N-dimethylformamide, washed with a large amount of water to obtain a precipitate, the precipitate was subjected to suction filtration, washed with water, and then dried under vacuum. After drying, 60 mg of a target zinc phthalocyanine complex was obtained, with a yield of 7%; a structural formula was as follows:

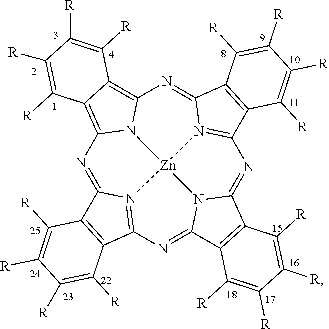

in the formula,

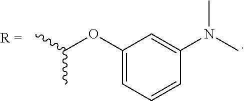 [SMILES: *Oc1cccc(N(C)C)c1]

The characterization data of the product were as follows: ¹HNMR (400 MHz,acetone-d6, ppm): δ7.09 (d,J=8.6 Hz,8H,Ar—H),6.95(s,7H,Ar—H),6.85-6.78(m, 8H,Ar—H),6.53-6.42(m,16H,Ar—H),6.31(s,J=8.0 Hz,9H,Ar—H),6.27-6.17(m,16H,Ar—H),2.81 (s, 54H, CH₃), 2.68 (s, 42H, CH₃).

HRMS (ESI): m/z calcd for C₁₆₀H₁₆₀N₂₄O₁₆ZnCl [M+Cl]—, 2775.1453; found 2775.1532.

IR (KBr, cm−1): 3445.87 (N—H); 1645.30, 1557.88, 1403.95 (C═C, C═N—).

### EXAMPLE 8

A synthesis method of a hexadeca-[3-(dimethylammonium)phenoxy]zinc phthalocyanine hexadecaiodate included the following steps:
1) according to the method in Example 7, the hexadeca-[3-(dimethylamino)phenoxy]zinc phthalocyanine complex was prepared; and
2) 0.36 mmol of the hexadeca-[3-(dimethylamino)phenoxy]zinc phthalocyanine complex and 3 mL of methyl iodide were added into 5 mL of chloroform, and subjected to a reaction by stirring at 25° C. for 12 h; after the reaction, a reaction solution was subjected to suction filtration, washed with chloroform until a filtrate was colorless, and a filter cake was collected and dried under vacuum to obtain 5 mg of a target product, with a yield of 46%; a structural formula was as follows:

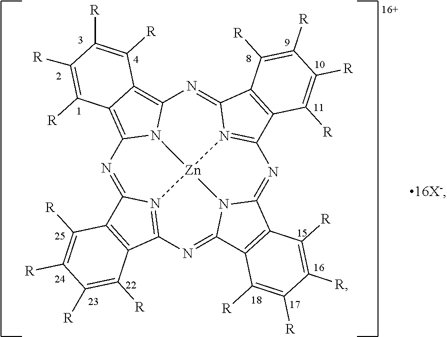

in the formula,

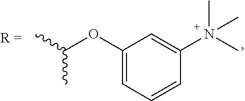

### and X is I.

The characterization data of the product were as follows: ¹ HNMR (400 MHz, DMSO-d6, ppm): δ8.02 (s,8H, Ar—H), 7.70-6.99 (m, 56H, Ar—H), 3.50 (s, 82H, CH₃), 3.47 (s, 62H, CH₃).

IR (KBr, cm⁻¹): 3432.34 (Ar—H); 2925.75 (—CH₃); 1609.72, 1491.44,1401.83 (C═C, C═N—); 1217.27 (Ar—O—Ar); 1123.61, 980.42, 509.94, 464.50,441.19 (Ar—H).

Elemental analysis (%) calcd for C₁₇₆H₂₀₈N₂₄O₁₆I₁₆Zn: C 42.18, H 4.18, N 6.71, found C 41.03, H 4.06, N 6.28.

### EXAMPLE 9

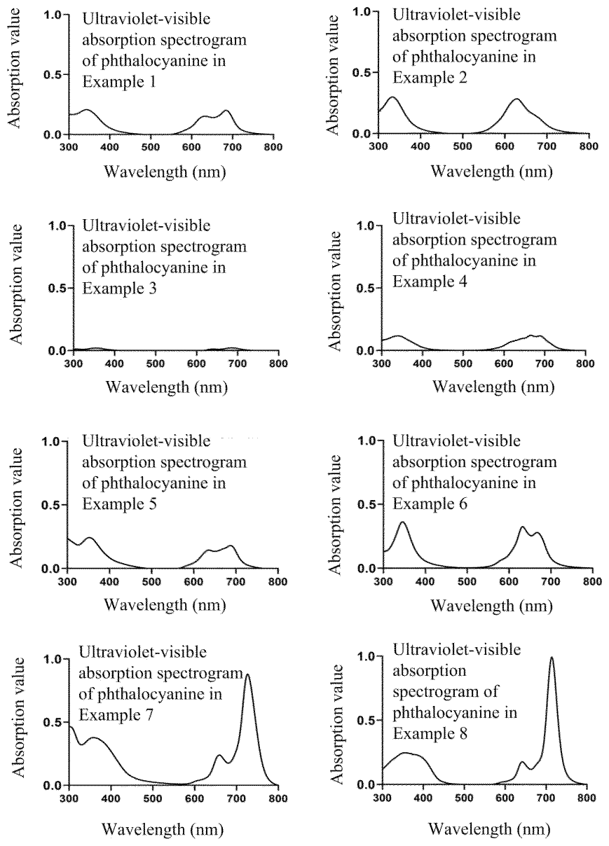

FIG. 1 showed an ultraviolet-visible absorption spectrum of zinc phthalocyanine complexes in each example in water, namely the 2,3-bis[3-(dimethylamino)phenoxy]zinc phthalocyanine complex of Example 1, the 2,3-bis[3-(trimethylammonium)phenoxy]zinc phthalocyanine diiodate of Example 2, the 1,2,3,4-bis[3-(dimethylamino)phenoxy]zinc phthalocyanine complex of Example 3, the 1,2,3,4-bis[3-(trimethylammonium)phenoxy]zinc phthalocyanine tetraiodate of Example 4, the octa-β-[3-(dimethylamino)phenoxy]zinc phthalocyanine complex of Example 5, the octa-β-[3-(trimethylammonium)phenoxy]zinc phthalocyanine octaiodate of Example 6, the hexadeca-[3-(dimethylamino)phenoxy]zinc phthalocyanine complex of Example 7, and the hexadeca-[3-(trimethylammonium)phenoxy]zinc phthalocyanine hexadecaiodate of Example 8.

It was seen from the figure that the Q bands of the zinc phthalocyanine complexes obtained in Examples 1 to 6 in water each were relatively wide and low, showing different degrees of aggregation; while only the Q bands of the zinc phthalocyanine complexes in Examples 7 and 8 in water were strong and sharp peaks. This demonstrated that the hexadeca-[3-(dimethylamino)phenoxy]zinc phthalocyanine complex of Example 7, and the hexadeca-[3-(trimethylammonium)phenoxy]zinc phthalocyanine hexadecaiodate of Example 8 were monomers in water, with maximum absorption wavelengths of 727 nm and 714 nm, respectively, which was beneficial to PDT.

### EXAMPLE 10

A test was conducted according to a method in the literature (Bioorganic & Medicinal Chemistry Letters, 2015, 25, 2386-2389). The hexadeca[3-(trimethylammonium)phenoxy]zinc phthalocyanine hexadecaiodate of Example 8 had a singlet oxygen yield of 0.44 in water; however, the products obtained in Example 2, Example 4, and Example 6 had abilities to generate singlet oxygen after photosensitization in water of 0.42, 0.19, and 0.14, respectively.

### EXAMPLE 11

A method for preparing a photosensitive medicament with the zinc phthalocyanine complex included: the zinc phthalocyanine complex was dissolved in water or a mixed solution of water and other substances, to obtain the photosensitive medicament with a certain concentration (where a concentration of the zinc phthalocyanine complex was not higher than that in a saturated solution of the zinc phthalocyanine complex); and an additive including an antioxidant, a buffer, and an isotonic agent was added to a prepared medicament solution to maintain a chemical stability and a biocompatibility of the photosensitive medicament. The mixed solution had not greater than 10% of the other substances by mass fraction; and the other substances were one or a mixture of two or more selected from the group consisting of a castor oil derivative (Cremophor EL), DMSO, ethanol, glycerol, N,N-dimethylformamide, polyethylene glycol 300 to polyethylene glycol 3000, cyclodextrin, glucose, Tween, and polyethylene glycol monostearate.

In the present disclosure, the zinc phthalocyanine complex dissolved in 5 wt % to 35 wt % of a DMSO aqueous solution was used as a pharmaceutical preparation for topical administration.

The zinc phthalocyanine complex was used to prepare photodynamic drugs, photosensitive medicaments, or photosensitizers, and also used in PDT, photodynamic diagnosis, or photodynamic disinfection, which was the same as the preparation method and use method of using zinc phthalocyanine complexes or porphyrin compounds not described in the present disclosure in the prior art. However, a suitable light source was required, and the suitable light source could be provided by an ordinary light source connected with a suitable filter, or by a laser with a specific wavelength; the suitable light source had a wavelength of 300 nm to 800 nm, preferably 680 nm to 730 nm.

### EXAMPLE 12

The dark toxicity and photodynamic anticancer effect of the 2,3-bis[3-(trimethyl ammonium)phenoxy]zinc phthalocyanine diiodate of Example 2, the 1,2,3,4-bis[3-(trimethylammonium)phenoxy]zinc phthalocyanine tetraiodate of Example 4, the octa-β-[3-(trimethylammonium)phenoxy]zinc phthalocyanine octaiodate of Example 6, and the hexadeca-[3-(trimethylammonium)phenoxy]zinc phthalocyanine hexadecaiodate of Example 8 on human liver cancer cells HepG2 were tested, respectively.

The main steps were as follows: the above photosensitive medicaments were separately dissolved in water to obtain 1 mM photosensitizers, and then diluted into a cell medium to prepare cell mediums containing zinc phthalocyanine complexes at different concentrations. The human liver cancer cells HepG2 were cultured in the mediums containing zinc phthalocyanine complexes at different concentrations for 2 h; the medium was discarded; and after washing with PBS, the cells were added into a new medium (without zinc phthalocyanine complexes). Illumination experiment group: the cells were irradiated with red light for 30 min at a power of irradiated light at 15 mW cm⁻² using red light with a wavelength greater than 600 nm as an excitation light source; non-illumination control group: the cells were placed in the dark for 30 min. After illumination or non-illumination, the cell viability was examined by MTT.

The red light with a wavelength greater than 610 nm was provided by a 500 W halogen lamp connected to an insulated water tank and an optical filter greater than 610 nm.

The results showed that without illumination, the zinc phthalocyanine complex obtained in Example 2, Example 4, Example 6, or Example 8 had no killing and growth inhibitory effect on HepG2 cells, indicating no dark toxicity; however, after illumination with red light, the zinc phthalocyanine complexes obtained in Example 2 and Example 4 each showed a high photodynamic anticancer activity. By examining a dose-effect relationship between the concentration of the zinc phthalocyanine complex obtained in Example 2, Example 4, Example 6, or Example 8 and the cell viability, a half maximal inhibitory concentration (IC₅₀, the concentration of drug required to kill 50% of cancer cells) was obtained under illumination. The results were 170 nM for the 2,3-bis[3-(trimethylammonium)phenoxy]zinc phthalocyanine diiodate of Example 2, 210 nM for the 1,2,3,4-bis[3-(trimethylammonium)phenoxy]zinc phthalocyanine tetraiodate of Example 4, 60 nM for the octa-β-[3-(trimethylammonium)phenoxy]zinc phthalocyanine octaiodate of Example 6, and 40 nM for the hexadeca-[3-(trimethylammonium)phenoxy]zinc phthalocyanine hexadecaiodate of Example 8, which were 1.23 times, 1.00 times, 1.50 times, and 2.25 times that of the same type of phthalocyanine (tetrakis-α-(2,4,6-tris(N,N,N-trimethylammonium methyl)-phenoxy)zinc phthalocyanine dodecaiodate). The hexadeca-[3-(trimethylammonium)phenoxy] zinc phthalocyanine hexadecaiodate showed a minimum IC₅₀ value, illustrating that the hexadeca ammonium-modified phthalocyanine of the present disclosure had a higher photodynamic activity.

### EXAMPLE 13

The 2,3-bis[3-(trimethylammonium)phenoxy]zinc phthalocyanine diiodate of Example 2, the 1,2,3,4-bis[3-(trimethylammonium)phenoxy]zinc phthalocyanine tetraiodate of Example 4, the octa-β-[3-(trimethylammonium)phenoxy]zinc phthalocyanine octaiodate of Example 6, and the hexadeca-[3-(trimethylammonium)phenoxy]zinc phthalocyanine hexadecaiodate of Example 8 each were dissolved in a physiological saline and diluted into solutions to be tested. A tumor-bearing (liver cancer H22) mouse model was established according to a method in the literature (ACS Applied Materials & Interfaces, 2019, 11(40), 36435-36443), 100 μL of each phthalocyanine solution (at an administration dosage: 0.8 mol·Kg⁻¹) was injected through the tail vein, and small animal live imaging was conducted within the next 24 h.

The results showed that the zinc phthalocyanine complex of Example 2 was distributed throughout the body immediately after intravenous injection, and then gradually accumulated in the tumor site, reaching the peak at 12 h; after intravenous injection of the zinc phthalocyanine complex of Example 6, the fluorescence of the tumor site reached its peak in about 12 h; after injecting the zinc phthalocyanine complex of Example 8, the tumor fluorescence reached its peak in 2 h, and the zinc phthalocyanine complex was basically scavenged by the body's metabolism in about 6 h. The zinc phthalocyanine complex of Example 4 had poor accumulation ability in tumor sites. This showed that the zinc phthalocyanine complexes obtained in Examples 2 and 6 were enriched to varying degrees in organs such as liver and spleen, with low metabolism; only the hexadeca[3-(trimethylammonium)phenoxy]zinc phthalocyanine hexadecaiodate of Example 8 exhibited excellent targeting ability and rapid metabolism in vivo.

### EXAMPLE 14

The zinc phthalocyanine complex obtained in Example 8 was dissolved in physiological saline and diluted to form a solution to be tested. 6 KM mice with subcutaneous tumors were taken, and each drug was divided into 4 groups (a medication+laser group, a medication group, a normal saline group, and a normal saline+laser group), with 5 mice in each group; when the tumor grew to (60-100) mm³, 100 μL of a phthalocyanine aqueous solution was injected intravenously at a dosage of 0.8 mol·Kg⁻¹. After 1 h, the mice were anesthetized and irradiated with laser of (685±5) nm (at a light intensity of 9.4 mW cm⁻² for 10 min). The mice were continued to be fed, and observed every other day; a weight of the mice was measured and a long diameter and a short diameter of the mice were measured with a vernier caliper for a total of 14 d.

The results showed that after 14 d of experiments, the tumors of the mice in the normal saline group increased by about 18 times, and the medication with hexadeca-[3-(trimethylammonium)phenoxy]zinc phthalocyanine hexadecaiodate+laser group had a tumor inhibition rate in mice reaching 98.7% (p<0.001), indicating that the hexadeca-[3-(trimethylammonium)phenoxy]zinc phthalocyanine hexadecaiodate had a desirable anti-tumor activity. The body weight of the mice in the treatment group showed a tendency to increase within 14 d, indicating that the hexadeca-[3-(trimethylammonium)phenoxy]zinc phthalocyanine hexadecaiodate had no obvious toxicity to mice, and had excellent biocompatibility.

### EXAMPLE 15

The dark toxicity and photodynamic antibacterial effect of the 2,3-bis[3-(trimethylammonium)phenoxy]zinc phthalocyanine diiodate of Example 2, the 1,2,3,4-bis[3-(trimethylammonium)phenoxy]zinc phthalocyanine tetraiodate of Example 4, the octa-β-[3-(trimethylammonium)phenoxy]zinc phthalocyanine octaiodate of Example 6, and the hexadeca-[3-(trimethylammonium)phenoxy]zinc phthalocyanine hexadecaiodate of Example 8 against Staphylococcus aureus were tested.

1 mM of the photosensitive medicament was diluted into PBS to prepare a PBS solutions containing zinc phthalocyanine complexes at different concentrations, and then Staphylococcus aureus was cultured in the PBS solutions containing zinc phthalocyanine complexes at different concentrations for 4 h. Illumination experiment group: the cells were irradiated with red light for 30 min at a power of irradiated light at 15 mW cm⁻² using red light with a wavelength greater than 610 nm as an excitation light source; non-illumination control group: the cells were placed in the dark for 30 min. A treated cell suspension was inoculated on a surface of a Luria-Bertani medium, cultured for 48 h, and the dark toxicity and photodynamic activity of the photosensitive medicament to Staphylococcus aureus were examined by the number of colonies.

The red light with a wavelength greater than 610 nm was provided by a 500 W halogen lamp connected to an insulated water tank and an optical filter greater than 610 nm.

The results showed that without illumination, the zinc phthalocyanine complex obtained in Example 2, Example 4, Example 6, or Example 8 had no killing and growth inhibitory effect on the Staphylococcus aureus, indicating no dark toxicity; however, after illumination with red light, the zinc phthalocyanine complexes obtained in Example 2 and Example 4 each showed a high photodynamic antibacterial activity. By examining a dose-effect relationship between the concentration of the zinc phthalocyanine complex obtained in Example 2, Example 4, Example 6, or Example 8 and the cell viability, an IC₉₀ value (a concentration of drug required to kill 90% of Staphylococcus aureus) was obtained under illumination. The results were 85 nM for the 2,3-bis[3-(trimethylammonium)phenoxy]zinc phthalocyanine diiodate of Example 2, 74 nM for the 1,2,3,4-bis[3-(trimethylammonium)phenoxy]zinc phthalocyanine tetraiodate of Example 4, 71 nM for the octa-β-[3-(trimethylammonium)phenoxy]zinc phthalocyanine octaiodate of Example 6, and 69 nM for the hexadeca-[3-(trimethylammonium)phenoxy]zinc phthalocyanine hexadecaiodate of Example 8, which were increased by 69 times, 80 times, 83 times, and 86 times compared with an IC₉₀ value (5.9 μM) of the commonly used photosensitizer MB, respectively, indicating that the hexadeca[3-(trimethylammonium)phenoxy]zinc phthalocyanine hexadecaiodate had an efficient photodynamic antibacterial effect.

### EXAMPLE 16

A photodynamic antibacterial effect of the hexadeca-[3-(trimethylammonium)phenoxy]zinc phthalocyanine hexadecaiodate of Example 8 was tested on mice infected with Staphylococcus aureus.

0.1 mL of a PBS solution containing Staphylococcus aureus colonies was inoculated subcutaneously on the right abdomen of depilated normal-grade KM female mice (weighed about 20 g), at a cell concentration of (1-2)×10⁶ CFU·mL⁻¹, and the mice could be treated two days after inoculation. 4 groups were set up, namely a hexadeca-[3-(trimethylammonium)phenoxy]zinc phthalocyanine hexadecaiodate+laser group, a hexadeca-[3-(trimethylammonium)phenoxy]zinc phthalocyanine hexadecaiodate group, a blank+laser group, and a blank group. Before treatment, a sterile absorbent pad of a wound dressing was soaked with 100 μL of the phthalocyanine aqueous solution (containing 1% CEL), applied to the wound of the treatment group, and fixed with a zinc oxide tape. The wound dressing was applied for 2 h each time, and then the dressing was changed once, and then continued for another 2 h. The blank group was given normal saline for applying. After 4 h, the illumination group was treated with laser at (685±5) nm (with a light intensity of 9.4 mW·cm⁻² for 5 min). The mice were observed and recorded for 15 d. After 15 d, the tissues at the infected site were excised, ground and extracted with 1 mL of a sterilized PBS to obtain a bacterial suspension, diluted by 50 times, spread on the surface of the corresponding Luria-Bertani medium, with parallelly 3 plates for each concentration, cultured in a 37° C. incubator, and then observed and counted after 24 h.

The results showed that the blank group, the blank+laser group, and the hexadeca-[3-(trimethylammonium)phenoxy]zinc phthalocyanine hexadecaiodate group each had obvious lumps, indicating that there was still Staphylococcus aureus infection in vivo. On the contrary, the skin of the hexadeca-[3-(trimethylammonium)phenoxy]zinc phthalocyanine hexadecaiodate+laser group had crusted, indicating that the infected Staphylococcus aureus had been killed and cleared.

In order to further evaluate the photodynamic antibacterial effect, the treated parts were excised, and the excised tissues were ground and cultured on the Luria-Bertani medium for 24 h. The hexadeca-[3-(trimethylammonium)phenoxy]zinc phthalocyanine hexadecaiodate+laser group could hardly see Staphylococcus aureus colonies, with an inhibition rate of 98%, while the other three groups showed a large number of the Staphylococcus aureus colonies. This further demonstrated that the hexadeca-[3-(trimethylammonium)phenoxy]zinc phthalocyanine hexadecaiodate had an efficient photodynamic antibacterial effect on superficial microbial infections, which was a promising antibacterial photosensitizer.

The above description of examples is merely provided to help understand the method of the present disclosure and a core idea thereof. It should be noted that, several improvements and modifications may be made by a person of ordinary skill in the art without departing from the principle of the present disclosure, and these improvements and modifications should also fall within the protection scope of the present disclosure. Various amendments to these examples are apparent to those of professional skill in the art, and the general principles defined herein may be implemented in other examples without departing from the spirit or scope of the present disclosure. Thus, the present disclosure is not limited to the examples shown herein but falls within the widest scope consistent with the principles and novel features disclosed herein.

1. A hexadeca amino- or hexadeca ammonium-modified zinc phthalocyanine, wherein the hexadeca amino-modified zinc phthalocyanine comprises 3-(dimethylamino)phenoxy substituent, the substituent is separately located at peripheral positions α and β of a phthalocyanine ring, namely positions 1, 2, 3, 4, 8, 9, 10, 11, 15, 16, 17, 18, 22, 23, 24, and 25; and the hexadeca amino-modified zinc phthalocyanine has a structural formula as follows: 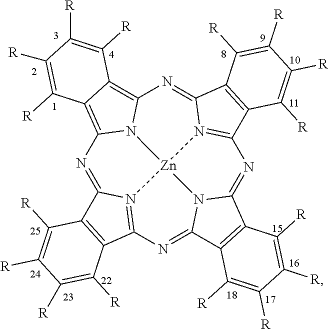
wherein in the formula, 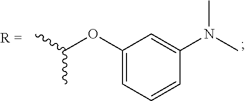 [SMILES: *Oc1cccc(N(C)C)c1]
and the hexadeca ammonium-modified zinc phthalocyanine comprises a 3-(trimethylammonium) phenoxy substituent, the substituent is separately located at peripheral positions α and β of a phthalocyanine ring, namely positions 1, 2, 3, 4, 8, 9, 10, 11, 15, 16, 17, 18, 22, 23, 24, and 25; and the hexadeca ammonium-modified zinc phthalocyanine has a structural formula as follows: 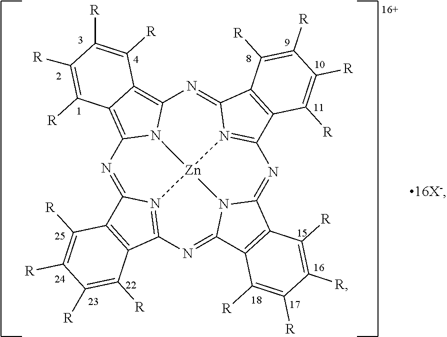
wherein in the formula, 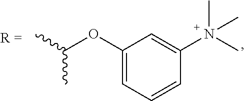
and X is selected from the group consisting of I and Br.

2. (canceled)

3. A preparation method of the hexadeca amino-modified zinc phthalocyanine according to claim 1, comprising the following steps:
1) preparation of 3,4,5,6-tetrakis[3-(dimethylamino)phenoxy]phthalonitrile: conducting a reaction by stirring at 110° C. for 18 h to 20 h with 3,4,5,6-tetrachlorophthalonitrile and N,N-dimethyl-3-aminophenol as a reactant using N,N-dimethylformamide as a solvent in the presence of potassium carbonate and nitrogen protection; monitoring the reaction by thin-layer chromatography, and terminating the reaction when the 3,4,5,6-tetrachlorophthalonitrile is consumed; and conducting purification by extraction, chromatography, or recrystallization to obtain the 3,4,5,6-tetrakis[3-(dimethylamino)phenoxy]phthalonitrile; and
2) preparation of the hexadeca amino-modified zinc phthalocyanine: using the 3,4,5,6-tetrakis[3-(dimethylamino)phenoxy]phthalonitrile as a raw material and methanol as a solvent, and adding zinc acetate to obtain a mixture; conducting a reaction by stirring on the mixture at 65° C. to 85° C. for 5 h to 6 h with 1,8-diazabicyclo[5.4.0]undec-7-ene as a catalyst; monitoring a reaction endpoint by the thin-layer chromatography to generate the hexadeca amino-modified zinc phthalocyanine; and conducting purification by a solvent method or the chromatography to obtain a target product.

4. A preparation method of the hexadeca ammonium-modified zinc phthalocyanine according to claim 1, comprising the following steps:
1) preparation of 3,4,5,6-tetrakis[3-(dimethylamino)phenoxy]phthalonitrile: conducting a reaction by stirring at 110° C. for 18 h to 20 h with 3,4,5,6-tetrachlorophthalonitrile and N,N-dimethyl-3-aminophenol as a reactant using N,N-dimethylformamide as a solvent in the presence of potassium carbonate and nitrogen protection; monitoring the reaction by thin-layer chromatography, and terminating the reaction when the 3,4,5,6-tetrachlorophthalonitrile is consumed; and conducting purification by extraction, chromatography, or recrystallization to obtain the 3,4,5,6-tetrakis[3-(dimethylamino)phenoxy]phthalonitrile; and
2) preparation of the hexadeca amino-modified zinc phthalocyanine: using the 3,4,5,6-tetrakis[3-(dimethylamino)phenoxy]phthalonitrile as a raw material and methanol as a solvent, and adding zinc acetate to obtain a mixture; conducting a reaction by stirring on the mixture at 65° C. to 85° C. for 5 h to 6 h with 1,8-diazabicyclo[5.4.0]undec-7-ene as a catalyst; monitoring a reaction endpoint by the thin-layer chromatography, and conducting purification by a solvent method or the chromatography to obtain the hexadeca amino-modified zinc phthalocyanine; and
3) preparation of hexadeca ammonium-modified zinc phthalocyanine: using the hexadeca amino-modified zinc phthalocyanine prepared in step 2) as a raw material and chloroform or N,N-dimethylformamide as a solvent, and adding methyl iodide or methyl bromide to obtain a mixture; conducting a reaction by stirring on the mixture at 15° C. to 35° C. for 6 h to 12 h to generate the hexadeca ammonium-modified zinc phthalocyanine, and conducting purification by the solvent method or the chromatography to obtain a target product.

5. The preparation method according to claim 3, wherein in step 1), the 3,4,5,6-tetrachlorophthalonitrile and the N,N-dimethyl-3-aminophenol are added at a molar ratio of 1:(5.5-6.0); 5 mL to 6 mL of the N,N-dimethylformamide is used for per mmol of the 3,4,5,6-tetrachlorophthalonitrile, and 7.5 mmol to 8 mmol of the potassium carbonate is used for per mmol of the 3,4,5,6-tetrachlorophthalonitrile.

6. The preparation method according to claim 3, wherein in step 2), the 3,4,5,6-tetrakis[3-(dimethylamino)phenoxy]phthalonitrile and the zinc acetate are added at a molar ratio of (2-4):1; 10 mL to 15 mL of the methanol is used for per mmol of the 3,4,5,6-tetrakis[3-(dimethylamino)phenoxy]phthalonitrile, and 3 mL to 4 mL of the 1,8-diazabicyclo[5.4.0]undec-7-ene is used for per mmol of the 3,4,5,6-tetrakis[3-(dimethylamino)phenoxy]phthalonitrile.

7. The preparation method according to claim 4, wherein in step 3), 0.8 mL to 2 mL of the methyl iodide or the methyl bromide is used for per 0.1 mmol of the hexadeca amino-modified zinc phthalocyanine, and 1.0 mL to 2.0 mL of the N,N-dimethylformamide or 1.0 mL to 2.0 mL of the chloroform is used for per 0.1 mmol of the hexadeca amino-modified zinc phthalocyanine.

8. A method for preparing a photosensitizer or a photodynamic drug or a photosensitive medicament or a photodynamic antibacterial material using the zinc phthalocyanine according to claim 1.

9. The method according to claim 8, wherein a method for preparing the photosensitive medicament with the zinc phthalocyanine comprises: dissolving the zinc phthalocyanine in water or a mixed solution of water and other substances to prepare the photosensitive medicament.

10. The method according to claim 9, wherein the mixed solution has not greater than 10% of other substances by mass fraction; and the other substances are one or a mixture of two or more selected from the group consisting of a castor oil derivative, DMSO, ethanol, glycerol, N,N-dimethylformamide, polyethylene glycol 300 to polyethylene glycol 3000, cyclodextrin, glucose, Tween, and polyethylene glycol monostearate.

11. The method according to claim 8, wherein the photosensitive medicament further comprises an additive; and the additive comprises one or more of an antioxidant, a buffer, and an isotonic agent.

12. The method according to claim 9, wherein when the photosensitive medicament is a pharmaceutical preparation for topical administration, a preparation method of the photosensitive medicament comprises: dissolving the zinc phthalocyanine in a permeable solvent, or injecting the zinc phthalocyanine into an ointment, a lotion, or a gel and stirring uniformly.

13. The method according to claim 12, wherein the permeable solvent is a DMSO aqueous solution with a mass fraction of 5 wt % to 35 wt %.

14. (canceled)

15. The method according to claim 9, wherein the antibacterial material is used for Staphylococcus aureus.

16. The preparation method according to claim 4, wherein in step 1), the 3,4,5,6-tetrachlorophthalonitrile and the N,N-dimethyl-3-aminophenol are added at a molar ratio of 1:(5.5-6.0); 5 mL to 6 mL of the N,N-dimethylformamide is used for per mmol of the 3,4,5,6-tetrachlorophthalonitrile, and 7.5 mmol to 8 mmol of the potassium carbonate is used for per mmol of the 3,4,5,6-tetrachlorophthalonitrile.

17. The preparation method according to claim 4, wherein in step 2), the 3,4,5,6-tetrakis[3-(dimethylamino)phenoxy]phthalonitrile and the zinc acetate are added at a molar ratio of (2-4):1; 10 mL to 15 mL of the methanol is used for per mmol of the 3,4,5,6-tetrakis[3-(dimethylamino)phenoxy]phthalonitrile, and 3 mL to 4 mL of the 1,8-diazabicyclo[5.4.0]undec-7-ene is used for per mmol of the 3,4,5,6-tetrakis[3-(dimethylamino)phenoxy]phthalonitrile.

18. The method according to claim 9, wherein the photosensitive medicament further comprises an additive; and the additive comprises one or more of an antioxidant, a buffer, and an isotonic agent.
